(19) United States
(12) Patent Application Publication
(10) Pub. No.: US 2026/0363640 A1
(43) Pub. Date: Nov. 19, 2026

(54) SYSTEMS AND METHODS FOR ENERGY BIN DOWNSAMPLING

(71) Applicants: GE Precision Healthcare LLC, Waukesha, WI (US); Marquette University, Milwaukee, WI (US); The Board of Trustees of the Leland Stanford Junior University, Stanford, CA (US)

(72) Inventors: Tal Gilat-Schmidt, Whitefish Bay (WI); Fredrik Grönberg, Stockholm (SE); Martin Sjölin, Stockholm (SE); Jiahua Fan, New Berlin, WI (US); Brian W. Thomsen, Menomonee Falls, WI (US); Jean-Baptiste Thibault, Waukesha, WI (US); Jiang Hsieh, Brookfield, WI (US); Mats Danielsson, Taby (SE); Yirong Yang, Stanford, CA (US); Norbert J. Pelc, Aptos, CA (US); Adam S. Wang, Palo Alto, CA (US)

(21) Appl. No.: 19/750,678

(22) Filed: Jul. 22, 2026

Related U.S. Application Data
(63) Continuation of application No. 18/692,210, filed on Sep. 19, 2022, now abandoned.
(60) Provisional application No. 63/245,607, filed on Sep. 17, 2021.
(60) Provisional application No. 63/309,160, filed on Feb. 11, 2022.

(51) Int. Cl. A61B 6/42; A61B 6/00; A61B 6/03; A61B 6/58; G01T 1/17

(52) U.S. Cl. CPC A61B 6/4241; A61B 6/032; A61B 6/5205; A61B 6/583; A61B 6/585; G01T 1/17

(57) ABSTRACT

Methods and systems are provided for downsampling detector data in a computed tomography imaging system. In an example, a method for a photon counting computed tomography (PCCT) system includes, during a scan of an imaging subject, obtaining detector data from a photon counting detector of the PCCT system, the detector data comprising, for each pixel or detector element of the photon counting detector, photon counts partitioned into a plurality of energy bins based on an energy imparted by each photon on the photon counting detector, applying a bin factor to the plurality of energy bins for each pixel to downsample the plurality of energy bins into a reduced number of energy bins, and reconstructing one or more images from the reduced number of energy bins.

### CROSS-REFERENCE TO RELATED APPLICATIONS

The present application is a continuation of U.S. application Ser. No. 18/692,210, filed on Mar. 14, 2024, which is a national stage application under 35 U.S.C. § 371(c) of PCT Application No. PCT/US2022/044002, filed on Sep. 19, 2022, which claims priority to U.S. Provisional Application No. 63/245,607, filed on Sep. 17, 2021, and U.S. Provisional Application No. 63/309,160, filed on Feb. 11, 2022. The entire contents of each of the above-identified applications are hereby incorporated by reference for all purposes.

### BACKGROUND

Embodiments of the subject matter disclosed herein relate to imaging systems and methods, and more particularly, to energy bin downsampling in computerized tomography (CT) imaging systems.

In computed tomography (CT) imaging systems, an electron beam generated by a cathode is directed towards a target within an X-ray source or X-ray tube. A fan-shaped or cone-shaped beam of X-rays produced by electrons colliding with the target is directed towards a subject, such as a patient. After being attenuated by the object, the X-rays impinge upon an array of X-ray detectors, generating an image. The quality of a CT image may be increased by using Photon Counting CT (PCCT), where the X-ray detectors are photon counting detectors, and photons are counted to provide spectral information.

### SUMMARY

In an example, a method for a photon counting computed tomography (PCCT) system includes, during a scan of an imaging subject, obtaining detector data from a photon counting detector of the PCCT system, the detector data comprising, for each pixel or detector element of the photon counting detector, photon counts partitioned into a plurality of energy bins based on an energy imparted by each photon that impinges on the photon counting detector, applying a bin factor to the plurality of energy bins for each pixel to downsample the plurality of energy bins into a reduced number of energy bins, and reconstructing one or more images from the reduced number of energy bins.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

Embodiments of the subject matter disclosed herein relate to imaging systems and methods, and more particularly, to energy bin downsampling in computerized tomography (CT) imaging systems.

### BRIEF DESCRIPTION OF THE DRAWINGS

Various aspects of this disclosure may be better understood upon reading the following detailed description and upon reference to the drawings in which:

FIG. 1 shows a pictorial view of a computed tomography (CT) imaging system, in accordance with one or more embodiments of the present disclosure;

FIG. 2 shows a block schematic diagram of an example CT imaging system, in accordance with one or more embodiments of the present disclosure;

FIG. 3 is a schematic diagram of an exemplary detector array of a PCCT system, in accordance with one or more embodiments of the present disclosure;

FIG. 4 is a flow chart illustrating a high-level method for energy bin downsampling, according to embodiments of the present disclosure;

FIG. 5 is a flow chart illustrating a method for identifying target bin combinations to be applied for downsampling energy bins, according to embodiments of the present disclosure;

FIG. 6 is a flow chart illustrating a method for identifying target weight sets to be applied for downsampling energy bins, according to embodiments of the present disclosure;

FIG. 7 is a flow chart illustrating a method for downsampling energy bin counts, according to embodiments of the present disclosure;

FIG. 8 shows example graphs of energy spectra in combined energy bins;

FIG. 9 shows an example graph of energy spectra in combined bins with only contiguous bins combined;

FIG. 10 shows example grayscale images of a subject generated with full energy bin counts and combined energy bin counts;

FIG. 11 shows an example graph of target weight sets applicable to downsample energy bin counts to two bins.

FIG. 12 shows an example graph of energy spectra of the two bins combined with the target weight sets of FIG. 11;

FIG. 13 shows example calcium basis images generated with full bin energy counts and summed weighted energy bin counts;

FIG. 14 shows example water basis images generated with full bin energy counts and summed weighted energy bin counts;

FIG. 15 schematically shows a process for iteratively combining bins to identify target bin combinations; and

FIG. 16 shows example weight sets that may be applied to downsample bin counts in a localized manner.

### DETAILED DESCRIPTION

This description and embodiments of the subject matter disclosed herein relate to methods and systems for downsampling data acquired via a photon counting computed tomography (PCCT) system. In computed tomography (CT) imaging systems, an X-ray source or X-ray tube emits an X-ray beam towards an object, such as a patient, and X-rays attenuated by the subject are detected by one or more detectors (e.g., a detector array) to generate projection data that is used to reconstruct one or more images. The X-ray detector or detector array typically includes a collimator for collimating X-ray beams received at the detector, a scintillator disposed adjacent to the collimator for converting X-rays to light energy, and photodiodes for receiving the light energy from the adjacent scintillator and producing electrical signals therefrom. The intensity of the attenuated X-ray beam radiation received at the detector array is typically dependent upon the attenuation of the X-ray beam by the patient. Each detector element of a detector array produces a separate electrical signal indicative of the attenuated beam received by each detector element. The electrical signals are transmitted to a data processing system for analysis. The data processing system processes the electrical signals to facilitate generation of an image. Generally, in CT systems the X-ray source and the detector array are rotated about a gantry within an imaging plane and around the patient, and images are generated from projection data at a plurality of views at different view angles. For example, for one rotation of the X-ray source, 1000 views may be generated by the CT system.

Such conventional CT imaging systems utilize detectors that convert radiographic energy into current signals that are integrated over a time period, then measured and ultimately digitized. However, a drawback of such detectors is their inability to provide data or feedback as to the number and/or energy of photons detected. That is, the light emitted by the scintillator is a function of both a number of X-rays impinged and an energy level of the X-rays. The photodiodes may not be capable of discriminating between the energy level or the photon count from the scintillation. For example, two scintillators may illuminate with equivalent intensity and, as such, provide equivalent output to their respective photodiodes. Yet, despite yielding an equivalent light output, the number of X-rays received by each scintillator may be different, and the intensity of the X-rays may be different.

In contrast, PCCT detectors may provide photon counting and/or energy discriminating feedback with high spatial resolution. PCCT detectors can be caused to operate in an X-ray counting mode, and also in an energy measurement mode of each X-ray event. While a number of materials may be used in the construction of a direct conversion energy discriminating detector, semiconductors have been shown to be one preferred material. Typical materials for such use includes Cadmium Zinc Telluride (CZT), Cadmium Telluride (CdTe) and Silicon (Si), which have a plurality of pixilated anodes attached thereto.

A drawback of photon counting detectors, however, is that they generate a relatively large amount of data due to the increased number of pixels and energy bins. For example, PCCT systems may have a plurality of energy bins (e.g., 5 or 8) that are determined by a comparator that may be part of a readout of a data acquisition system (DAS). A system having many energy bins may be formed with multiple comparators in the readout DAS. Each comparator may be set to trigger above a set level of energy that results in accumulation on a register of the number of photons above a corresponding X-ray energy level. Because the detector arrays rotate, the collected data are transmitted to a data processing computer for image reconstruction through a slip ring, which has a limited bandwidth. Thus, initial image reconstruction may be delayed due to the relatively long amount of time it may take to transmit the data across the slip ring.

Thus, systems and methods are proposed herein that downsample the detector data into fewer energy bins, e.g., 2 or 3 bins rather than the full 5 or 8 bins, thereby reducing the amount of data that is transmitted across the slip ring. By downsampling the detector energy-bin data, images may be reconstructed and presented for review relatively quickly, which may aid clinical decision-making. In some examples, the detector data may be downsampled by combining energy-bin counts (also referred to herein as simply combining bins) according to a target bin combination identified during system calibration. The target bin combination may be identified that combines the full number of bins (e.g., 8) into 2 or 3 bins with minimum compromise in image quality by selecting the bin combination that maximizes a quality metric such as contrast to noise ratio. In other examples, the detector data may be downsampled by forming two or more weighted sums of the energy bins. Each weighted sum may be formed by applying a respective set of bin weights to the full bins and summing the weighted bins. Each set of bin weights may be identified empirically during system calibration, e.g., by selecting weights that minimize the ratio between the Cramér-Rao lower bound (CRLB) of basis material thickness estimates from the weighted sums and that from the original binned counts for a large range of different object materials and sizes. For each embodiment, the downsampled bin counts may be used to generate virtual monoenergetic images (VMI) and/or perform material decomposition (MD).

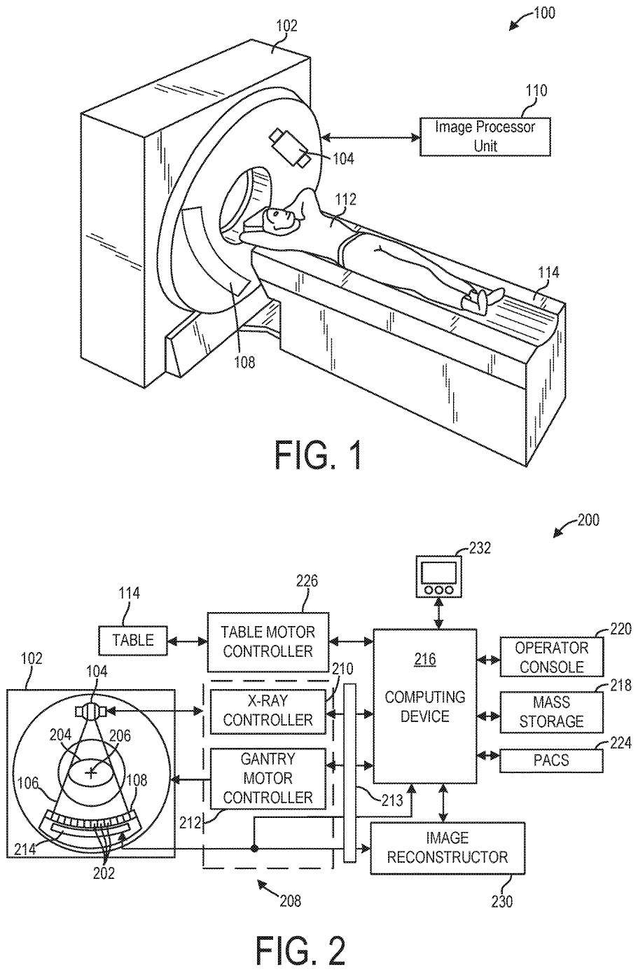

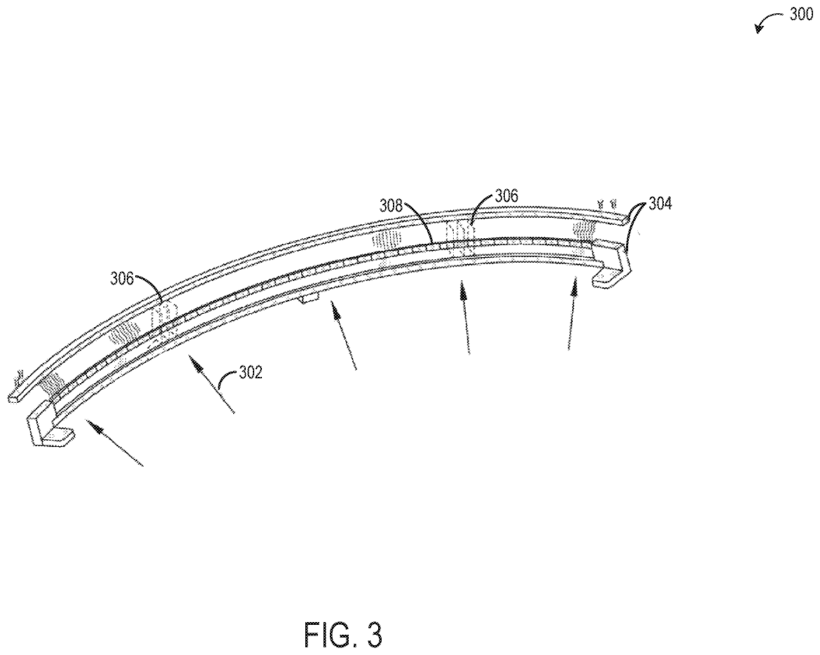

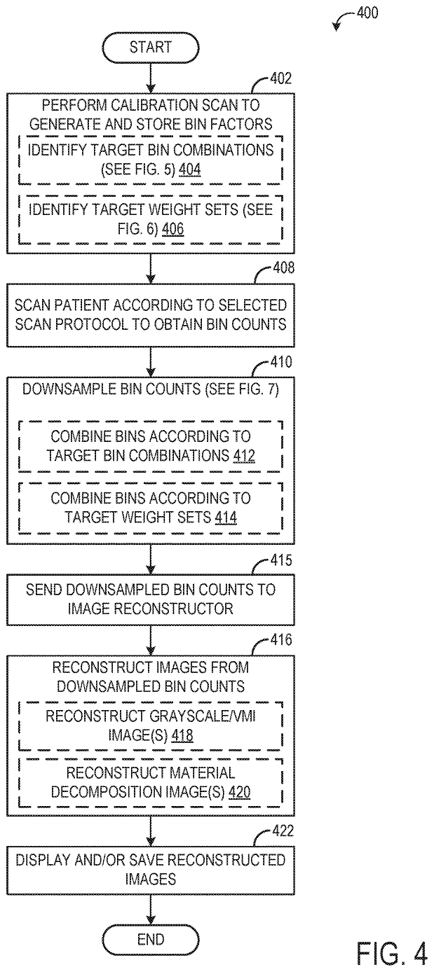

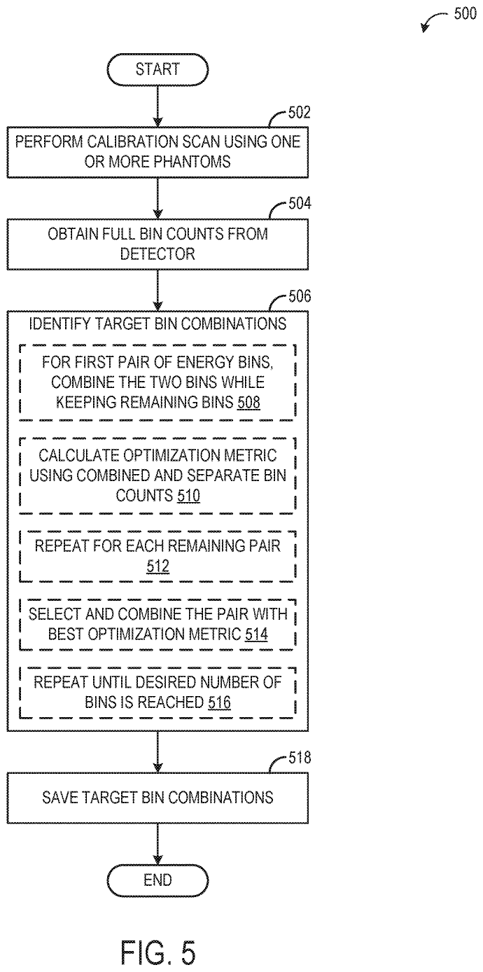

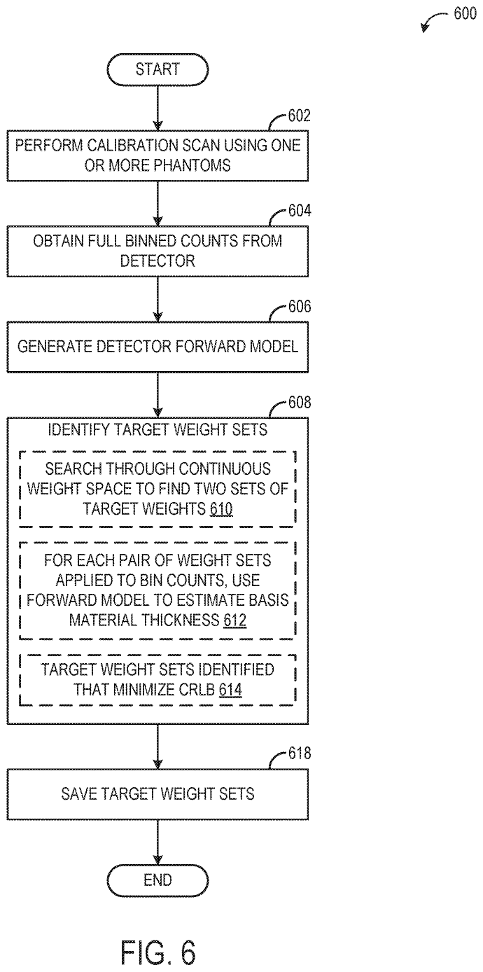

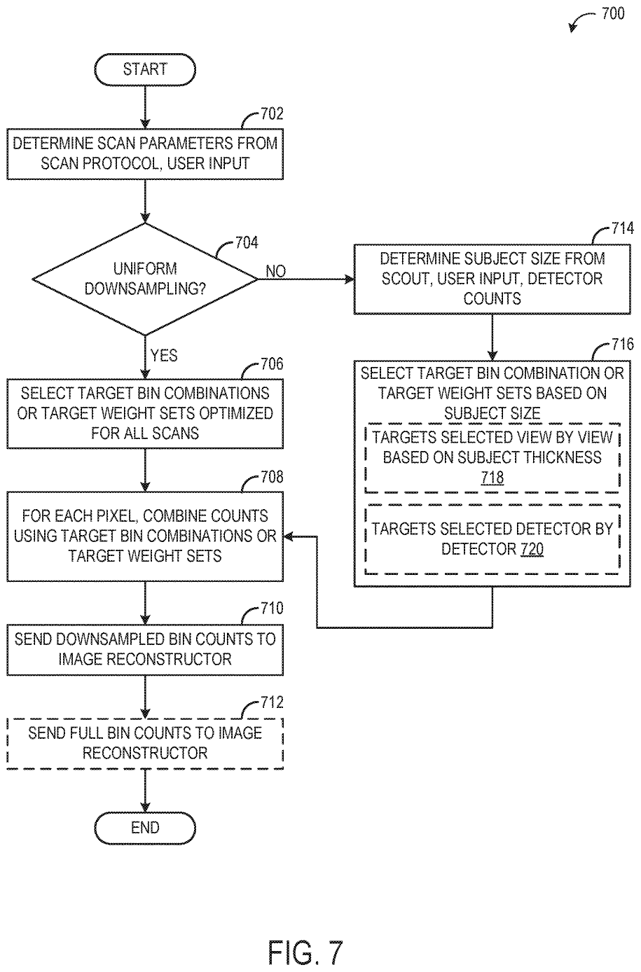

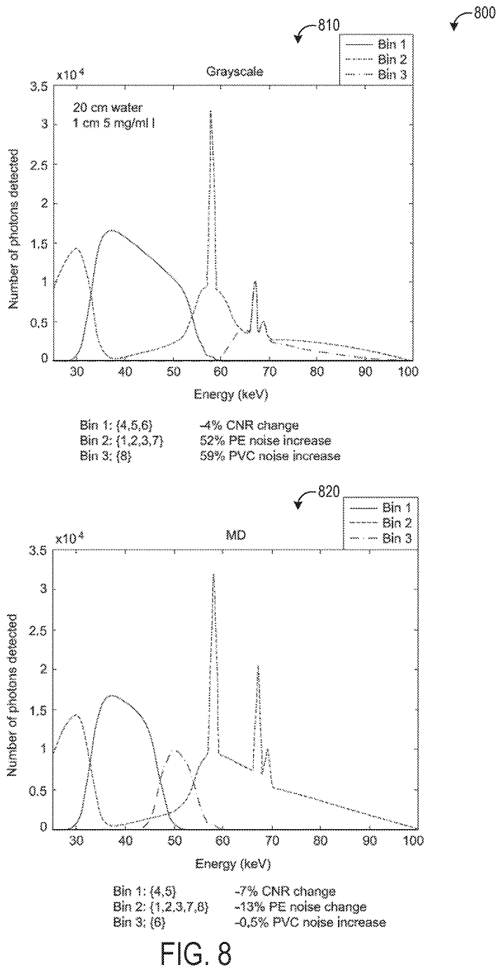

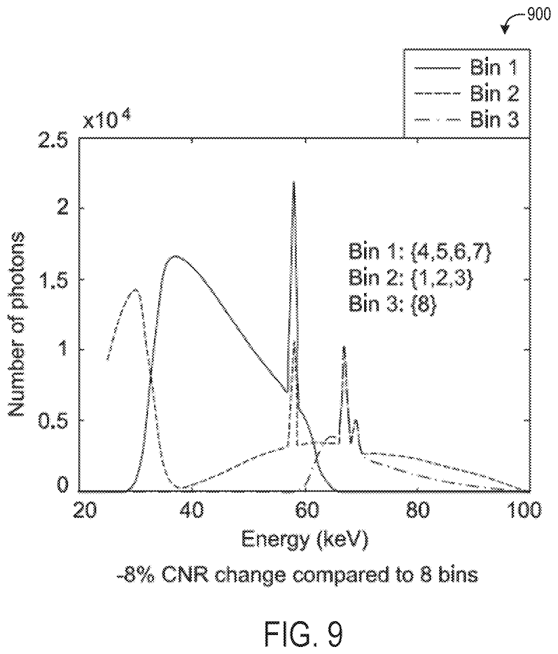

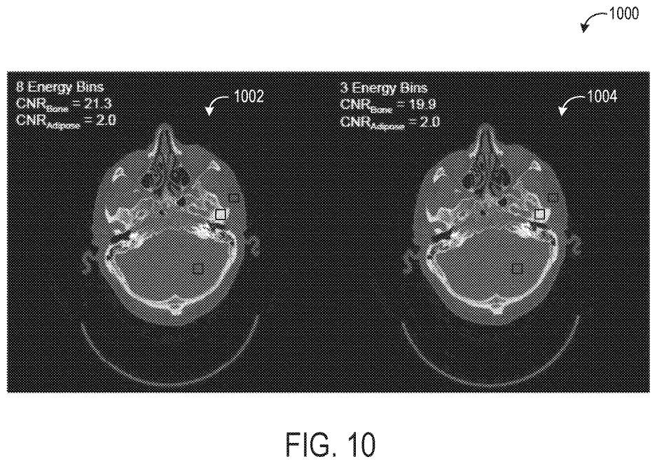

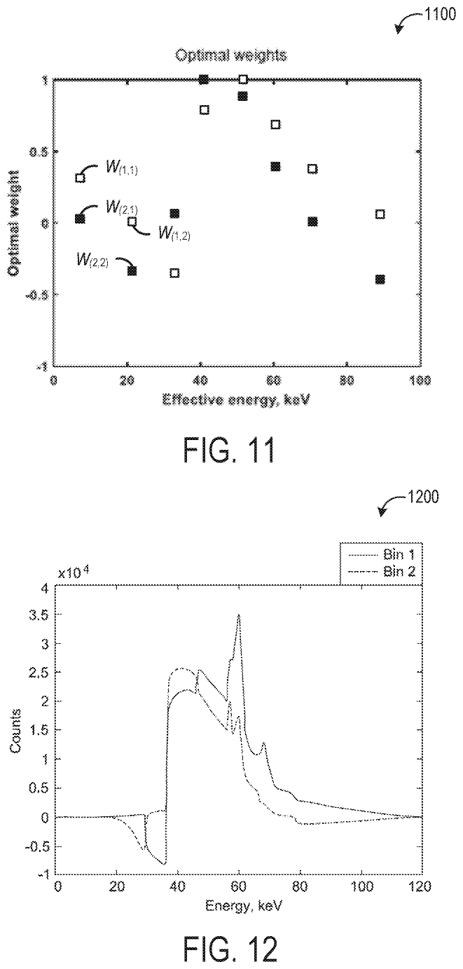

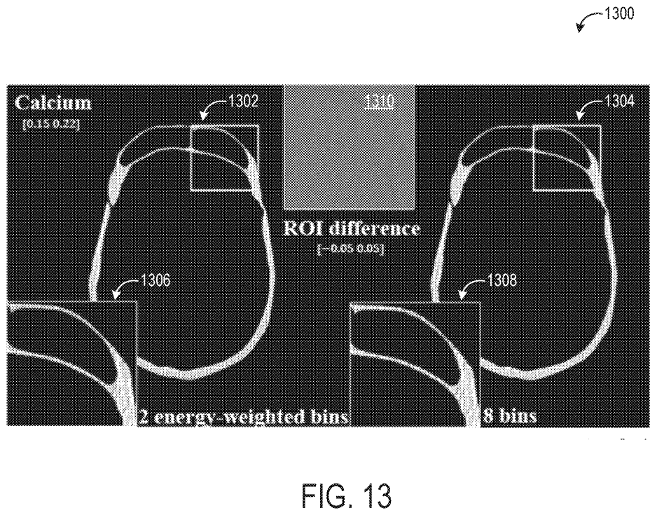

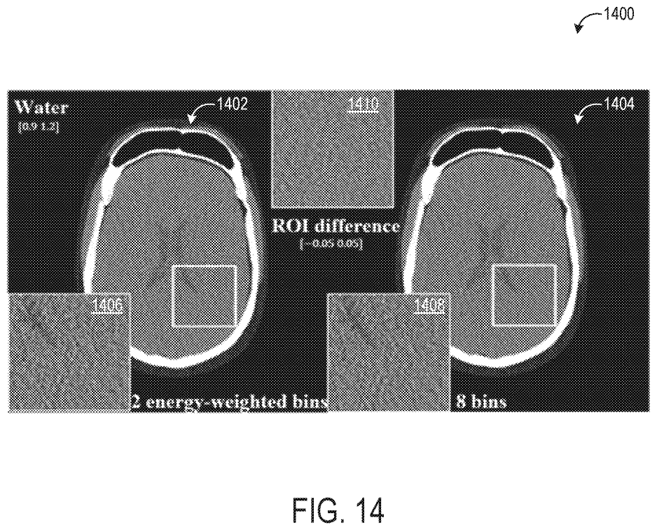

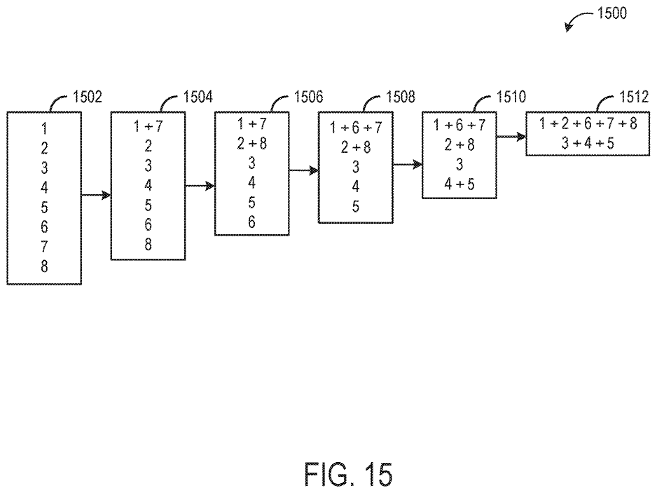

An example of a PCCT system that may be used to perform imaging scans in accordance with the present techniques is provided in FIGS. 1 and 2. FIG. 3 shows an example detector array of the PCCT system, where photons of X-rays directed at a subject by an X-ray source are counted by detectors of the detector array. The counted photons may be partitioned into bins and the bins may be downsampled according to the method shown in FIG. 4, which includes identifying target bin combinations (according to the method of FIG. 5 and as illustrated schematically in FIG. 15) or target weight sets (according to the method of FIG. 6) during calibration of the PCCT system and then applying the target bin combinations or target weight sets during a scan (according to the method of FIG. 7). When selecting target bin combinations during calibration, the bin combinations may be optimized for VMI or MD, which may result in different bin combinations, as shown by FIG. 8, and may not unduly compromise image quality, as shown by FIG. 10. As shown by FIG. 9, allowing for non-contiguous bins to be combined may provide higher image quality than simply reducing the number of energy bins. Example target weight sets for forming weighted sums of bins and corresponding energy spectra are shown in FIGS. 11, 12, and 16, which likewise do not unduly compromise image quality, as shown by FIGS. 13 and 14.

FIG. 1 illustrates an exemplary PCCT system 100 configured for CT imaging with photon counting detectors. Particularly, the PCCT system 100 is configured to image a subject 112 such as a patient, an inanimate object, one or more manufactured parts, and/or foreign objects such as dental implants, stents, and/or contrast agents present within the body. The PCCT system 100 includes a gantry 102, which in turn, may further include at least one X-ray source 104 configured to project a beam of X-ray radiation 106 (see FIG. 2) for use in imaging the subject 112 laying on a table 114. Specifically, the X-ray source 104 is configured to project the X-ray radiation beams 106 towards a detector array 108 positioned on the opposite side of the gantry 102. Although FIG. 1 depicts a single X-ray source 104, in certain embodiments, multiple X-ray sources and detectors may be employed to project a plurality of X-ray radiation beams for acquiring projection data at the same or different energy levels corresponding to the patient. In some embodiments, the X-ray source 104 may enable dual-energy gemstone spectral imaging (GSI) by rapid peak kilovoltage (kVp) switching. In the embodiments described herein, the X-ray detector employed is a photon counting detector which is capable of differentiating X-ray photons of different energies.

In certain embodiments, the PCCT system 100 further includes an image processor unit 110 configured to reconstruct images of a target volume of the subject 112 using an iterative or analytic image reconstruction method. For example, the image processor unit 110 may use an analytic image reconstruction approach such as filtered back projection (FBP) to reconstruct images of a target volume of the patient. As another example, the image processor unit 110 may use an iterative image reconstruction approach such as advanced statistical iterative reconstruction (ASIR), conjugate gradient (CG), maximum likelihood expectation maximization (MLEM), model-based iterative reconstruction (MBIR), and so on to reconstruct images of a target volume of the subject 112. In some examples the image processor unit 110 may use both an analytic image reconstruction approach such as FBP in addition to an iterative image reconstruction approach.

In some CT imaging system configurations, an X-ray source projects a cone-shaped X-ray radiation beam which is defined with respect to an X-Y-Z Cartesian coordinate system and generally referred to as an “imaging volume.” The X-ray radiation beam passes through an object being imaged, such as the patient or subject. The X-ray radiation beam, after being attenuated by the object, impinges upon an array of detector elements. The intensity of the attenuated X-ray radiation beam received at the detector array is dependent upon the attenuation of an X-ray radiation beam by the object. Each detector element of the array produces a separate electrical signal that is a measurement of the X-ray beam attenuation at the detector location. The attenuation measurements from all the detector elements are acquired separately to produce a transmission profile.

In some CT systems, the X-ray source and the detector array are rotated with a gantry within the imaging volume and around the object to be imaged such that an angle at which the X-ray beam intersects the object constantly changes. A group of X-ray radiation attenuation measurements, e.g., projection data, from the detector array at one gantry angle is referred to as a “view.” A “scan” of the object includes a set of views made at different gantry angles, or view angles, during one revolution of the X-ray source and detector.

FIG. 2 illustrates an exemplary imaging system 200 similar to the PCCT system 100 of FIG. 1. In accordance with aspects of the present disclosure, the imaging system 200 is configured for imaging a subject 204 (e.g., the subject 112 of FIG. 1). In one embodiment, the imaging system 200 includes the detector array 108 (see FIG. 1). The detector array 108 further includes a plurality of detector elements 202 that together sense the X-ray radiation beam 106 (see FIG. 2) that passes through the subject 204 (such as a patient) to acquire corresponding projection data. In some embodiments, the detector array 108 may be fabricated in a multi-slice configuration including the plurality of rows of cells or detector elements 202, where one or more additional rows of the detector elements 202 are arranged in a parallel configuration for acquiring the projection data. The detector elements 202 may also be referred to as pixels or detector pixels.

In certain embodiments, the imaging system 200 is configured to traverse different angular positions around the subject 204 for acquiring desired projection data. Accordingly, the gantry 102 and the components mounted thereon may be configured to rotate about a center of rotation 206 for acquiring the projection data, for example, at different energy levels. Alternatively, in embodiments where a projection angle relative to the subject 204 varies as a function of time, the mounted components may be configured to move along a general curve rather than along a segment of a circle.

As the X-ray source 104 and the detector array 108 rotate, the detector array 108 collects data of the attenuated X-ray beams. The data collected by the detector array 108 undergoes pre-processing and calibration to condition the data to represent the line integrals of the attenuation coefficients of the scanned subject 204. The processed data are commonly called projections. In some examples, the individual detectors or detector elements 202 of the detector array 108 may include photon counting detectors which register the interactions of individual photons into one or more energy bins.

The acquired sets of projection data may be used for basis material decomposition (BMD). During BMD, the measured projections are converted to a set of material-density projections. The material-density projections may be reconstructed to form a set of material-density maps or images of each respective basis material, such as bone, soft tissue, and/or contrast agent maps. The density maps or images may be, in turn, associated to form a 3D volumetric image of the basis material, for example, bone, soft tissue, and/or contrast agent, in the imaged volume.

Once reconstructed, the basis material image produced by the imaging system 200 reveals internal features of the subject 204, expressed in the densities of two basis materials. The density image may be displayed to show these features. In traditional approaches to diagnosis of medical conditions, such as disease states, and more generally of medical events, a radiologist or physician would consider a hard copy or display of the density image to discern characteristic features of interest. Such features might include lesions, sizes and shapes of particular anatomies or organs, and other features that would be discernable in the image based upon the skill and knowledge of the individual practitioner.

In one embodiment, the imaging system 200 includes a control mechanism 208 to control movement of the components such as rotation of the gantry 102 and the operation of the X-ray source 104. In certain embodiments, the control mechanism 208 further includes an X-ray controller 210 configured to provide power and timing signals to the X-ray source 104. Additionally, the control mechanism 208 includes a gantry motor controller 212 configured to control a rotational speed and/or position of the gantry 102 based on imaging requirements.

In certain embodiments, the control mechanism 208 further includes a data acquisition system (DAS) 214 configured to sample analog data received from the detector elements 202 and convert the analog data to digital signals for subsequent processing. The DAS 214 may be further configured to selectively aggregate data from a subset of the detector elements 202 into so-called macro-detectors. The data sampled and digitized by the DAS 214 is transmitted to a computer or computing device 216 via a slip ring. In one example, the computing device 216 stores the data in a storage device or mass storage 218. The storage device 218, for example, may be any type of non-transitory memory and may include a hard disk drive, a floppy disk drive, a compact disk-read/write (CD-R/W) drive, a Digital Versatile Disc (DVD) drive, a flash drive, and/or a solid-state storage drive.

Additionally, the computing device 216 provides commands and parameters to one or more of the DAS 214, the X-ray controller 210, and the gantry motor controller 212 for controlling system operations such as data acquisition and/or processing. In certain embodiments, the computing device 216 controls system operations based on operator input. The computing device 216 receives the operator input, for example, including commands and/or scanning parameters via an operator console 220 operatively coupled to the computing device 216. The operator console 220 may include a keyboard (not shown) or a touchscreen to allow the operator to specify the commands and/or scanning parameters.

Although FIG. 2 illustrates one operator console 220, more than one operator console may be coupled to the imaging system 200, for example, for inputting or outputting system parameters, requesting examinations, plotting data, and/or viewing images. Further, in certain embodiments, the imaging system 200 may be coupled to multiple displays, printers, workstations, and/or similar devices located either locally or remotely, for example, within an institution or hospital, or in an entirely different location via one or more configurable wired and/or wireless networks such as the Internet and/or virtual private networks, wireless telephone networks, wireless local area networks, wired local area networks, wireless wide area networks, wired wide area networks, etc.

In one embodiment, for example, the imaging system 200 either includes, or is coupled to, a picture archiving and communications system (PACS) 224. In an exemplary implementation, the PACS 224 is further coupled to a remote system such as a radiology department information system, hospital information system, and/or to an internal or external network (not shown) to allow operators at different locations to supply commands and parameters and/or gain access to the image data.

The computing device 216 uses the operator-supplied and/or system-defined commands and parameters to operate a table motor controller 226, which in turn, may control a table 114 which may be a motorized table. Specifically, the table motor controller 226 may move the table 114 for appropriately positioning the subject 204 in the gantry 102 for acquiring projection data corresponding to the target volume of the subject 204.

As previously noted, the DAS 214 samples and digitizes the projection data acquired by the detector elements 202. Subsequently, an image reconstructor 230 uses the sampled and digitized X-ray data to perform high-speed reconstruction. Although FIG. 2 illustrates the image reconstructor 230 as a separate entity, in certain embodiments, the image reconstructor 230 may form part of the computing device 216. Alternatively, the image reconstructor 230 may be absent from the imaging system 200 and instead the computing device 216 may perform one or more functions of the image reconstructor 230. Moreover, the image reconstructor 230 may be located locally or remotely, and may be operatively connected to the imaging system 200 using a wired or wireless network. Particularly, one exemplary embodiment may use computing resources in a “cloud” network cluster for the image reconstructor 230.

In one embodiment, the image reconstructor 230 stores the images reconstructed in the storage device 218. Alternatively, the image reconstructor 230 may transmit the reconstructed images to the computing device 216 for generating useful patient information for diagnosis and evaluation. In certain embodiments, the computing device 216 may transmit the reconstructed images and/or the patient information to a display or display device 232 communicatively coupled to the computing device 216 and/or the image reconstructor 230. In some embodiments, the reconstructed images may be transmitted from the computing device 216 or the image reconstructor 230 to the storage device 218 for short-term or long-term storage.

Information may be transmitted between the components residing in the gantry 102 and external devices (such as the computing device 216 and/or image reconstructor 230) via a slip ring 213, which facilitates electronic communication across the rotating gantry.

Referring now to FIG. 3, a PCCT photon counting detector array 300 is shown, which may be a non-limiting example of detector array 108 of FIG. 2. Detector array 300 includes rails 304 having collimating blades or plates 306 placed therebetween. Plates 306 are positioned to collimate X-rays 302 before such beams impinge upon a plurality of detector modules 308 of detector array 300, which may be arranged between the plates 306. As an example, detector array 300 may include 57 detector modules 308, each detector module 308 having an array size of 64×16 of detector elements (e.g., pixels). As a result, detector array 300 would have 64 rows and 912 columns (16 pixels×57 detector modules), allowing for 64 simultaneous slices of data to be collected with each gantry rotation (e.g., the gantry 102 of FIG. 1).

As described above, each detector element of each detector module 308 may be designed to directly convert radiographic energy to electrical signals containing energy discriminatory or photon count data. For example, when a photon impinges upon a detector element of a detector module 308, a charge may be generated within a semiconductor layer of the detector element that is proportional to the energy of the photon. A comparator may compare the voltage of the generated charge to one or more thresholds and increment a count of a bin (of a plurality of bins) based on the voltage relative to the one or more thresholds. The plurality of bins may include 8 bins, for example, with energy thresholds configured for optimal material decomposition performance.

Thus, the X-ray beam may generate multiple photon counts (e.g., one or more counts for each energy bin) for each detector element, resulting in substantially more data than that generated by integrating detectors. Generating images from the data collected with photon counting detectors may be time-consuming, which may delay image review. Further, transmitting the data from the DAS to an external computing device for image reconstruction may be challenging, as the rotating gantry necessitates a slip ring to transmit the data, which has a limited bandwidth and thus may limit the amount of data that can be transmitted. Accordingly, downsampling methods are provided herein to downsample the amount of data to be transmitted via the slip ring and/or used in image reconstruction.

In a first example, the energy bin downsampling may include combining the energy bins into two or three combined bins, with the bin combinations determined with the use of calibration transmission measurements to determine optimal combinations of bins to maintain image quality (for generating grayscale images and/or material decomposition images). To identify the bin combinations during calibration, at each iteration, a downsampling optimization method evaluates the combination of all pairs of energy bins and selects the combination that maximizes an image quality metric. Downsampling continues iteratively while the image quality metric is maintained. The image quality metric may be a contrast to noise ratio (CNR) between a measurement with and without a contrast element, but other metrics are possible, such as material decomposition basis noise. Additional details about energy bin downsampling using target bin combinations is provided below with respect to FIGS. 4, 5, and 7-9.

In a second example, the energy bin downsampling may include forming two or three weighted sums of the energy bins that include substantially the same amount of information as the original binned counts by applying two (or three) sets of weights to the energy bin counts. Additional details about energy bin downsampling to form two or more weighted sums is provided below with respect to FIGS. 4, 6, 7, 10, and 11.

Either example for downsampling the energy bins may be applied to perform material decomposition and/or generate virtual monoenergetic images. The energy bin downsampling described herein may significantly reduce the data size that needs to be transmitted through the slip ring without heavy computational loads or substantial loss of spectral information, which may result in faster processing/reconstruction times.

Turning now to FIG. 4, it shows a method 400 for performing a scan using energy bin downsampling. Method 400 may be carried out according to instructions stored in memory of one or more controllers or computing devices included as part of and/or operatively coupled to a CT imaging system, such as DAS 214, X-ray controller 210, image reconstructor 230, and/or computing device 216.

At 402, method 400 includes performing one or more calibration scans to generate and store bin factors. The bin factors may include bin combinations or weights that may be applied in order to compress/downsample energy bins during a subsequent scan on an imaging target such as a patient. Thus, in some examples, the calibration scan(s) may be performed to identify one or more target bin combinations, as indicated at 404. Each target bin combination may specify which contiguous or non-contiguous bins should be combined to reduce the full number of energy bins down to two or three combined bins. Additional details of identifying the target bin combinations are provided below with respect to FIG. 5. Briefly, during the calibration scan(s), detector data may be obtained while scanning object(s) of known size and composition, referred to as phantoms. The detector data may include photon counts partitioned into bins based on the energy of each photon, such as eight or five energy bins depending on the configuration of the detector. An iterative bin combination process may be applied to identify the bin combinations that optimize an image quality metric, such as CNR or material decomposition basis noise, while reducing the amount of transmitted and processed data. The identified bin combinations may be saved as the bin factor.

In other examples, the calibration scan(s) may be performed to identify one or more target weight sets, as indicated at 406. Each target weight set may specify a weight to apply to each energy bin, and the weighted bins may be summed to reduce the full number of energy bins down to two or three summed bins. Additional details of identifying the target weight sets are provided below with respect to FIG. 6. Briefly, a search may be performed using the detector data collected during the calibration scan to identify two or three weight sets that minimize variance of basis material thickness estimates from the weighted sums that is comparable to that from the original binned counts. The identified weight sets may be saved as the bin factors.

At 408, method 400 includes scanning a patient according to a selected scan protocol to obtain detector data including bin counts. The detector data may include, for each pixel of the photon counting detector (e.g., for each detector element 202), photon counts partitioned into a plurality of energy bins based on an energy imparted by each photon on the photon counting detector, which is referred to as bin counts herein. During the patient scan, the X-ray source (e.g., X-ray source 104 of FIGS. 1 and 2) of the CT imaging system may be controlled to emit X-rays according to a scan prescription set forth by the selected scan protocol (e.g., with a set X-ray source or X-ray tube current and voltage). Detector data may be obtained from the detector array (e.g., detector array 108 of FIGS. 1 and 2) as the X-ray source and detector array rotate around the patient, resulting in a plurality of views of detector data being obtained. For each view, a photon count for each energy bin for each pixel or detector element of the detector array is generated (e.g., during readout of the detector array by DAS 214). For example, in detector configurations with eight energy bins, eight photon counts may be generated for each pixel or detector element of the detector array and for each view. In this way, the output of the detector array may be referred to as the bin counts, as the photon counts are partitioned into energy bins based on the energy of each photon that impinges on the detector array. The number of energy bins may be based on the configuration of the detector. For example, silicon detectors may be configured to differentiate photon energy into 8 energy bins, while cadmium telluride detectors may be configured to differentiate photon energy into 5 bins. The energy thresholds that define the energy bins may be determined during a calibration phase and/or may be based on the specific scan protocol. For example, the energy thresholds may be determined to optimize material basis decomposition and/or to maximize detected spectral information for a given incident spectrum emitted by the X-ray source. In a non-limiting example, the energy bin thresholds may be 4, 14, 30, 37, 47, 58, 67, and 79 keV for an 8 bin detector, or 10, 34, 50, 62, and 76 keV for a 5 bin detector.

At 410, the bin counts are downsampled using the stored bin factors. As indicated at 412, downsampling the bin counts may include combining energy bins according to the target bin combinations determined at 404. For example, if the target bin combination indicates that bins 1, 2, 3, and 7 should be combined into a first bin, bins 4, 5, and 6 should be combined into a second bin, and bin 8 should be a third bin, the bin counts (for each pixel) may be combined so that bins 1, 2, 3, and 7 are combined; bins 4, 5, and 6 are combined; and bin 8 is left as is. In other examples, as indicated at 414, the bin counts may be combined according to the target weight sets determined at 406. For example, the target weight sets may include a first target weight set and a second target weight set. Each target weight set may include a respective weight to be applied to each energy bin count. For the first target weight set, each weight may be applied to the respective energy bin, which may adjust the counts of one or more or all of the energy bins. For example, the first target weight set may indicate that bin 1 is to be weighted by 0.5 while bin 2 is to be weighted by 1. Applying the weights may result in the count for bin 1 being halved while the count for bin 2 is not changed. After applying the weights from the first target weight set, the weighted counts may be summed to form a first weighted bin. The same process may be performed for the second target weight set (e.g., where the second target weight set is applied to the original, full bin counts to form a second set of weighted counts that are then summed), such that two weighted bins are formed. Additional details about downsampling the bin counts are presented below with respect to FIG. 7.

At 415, the downsampled bin counts are sent to an image reconstructor or another suitable computing device configured to reconstruct one or more images from the downsampled bin counts. The downsampled bin counts may be sent from the DAS of the CT scanner to the image reconstructor via a slip ring of the CT scanner.

At 416, method 400 includes reconstructing one or more images from the downsampled bin counts. The images may be reconstructed by the image reconstructor or other suitable computing device. Reconstructing the one or more images may include reconstructing one or more grayscale (e.g., virtual monoenergetic) images from the downsampled bin counts, as indicated at 418. To reconstruct a VMI, the downsampled bin counts may be decomposed into selected basis materials or attenuation components (e.g., Compton and photoelectric) using maximum likelihood estimation (MLE), least-squared, polynomial fitting, neural networks, or other suitable decomposition methods, and then material decomposition (MD) images may be reconstructed using filtered backprojection or another suitable reconstruction technique. A VMI at a selected energy (e.g., 68 keV) may be formed by linear combination of the reconstructed images.

Additionally or alternatively, reconstructing the one or more images may include reconstructing one or more MD images, as indicated at 420. To reconstruct MD images, the downsampled bin counts may be decomposed into selected basis materials (e.g., calcium and water) using a suitable decomposition method as explained above (e.g., MLE or least-squared) and then MD images may be reconstructed using a suitable reconstruction technique such as filtered backprojection. At 422, the reconstructed images are displayed on a display device and/or saved in memory (e.g., in a PACS as part of a patient exam).

Thus, a plurality of energy bin counts (such as 5 or 8 energy bins) may be compressed into a reduced number of energy bins (such as 2 or 3) and sent for image reconstruction, which may expedite the reconstruction of at least initial images during a CT exam. Once the compressed detector data (e.g., the reduced number of energy bins) is sent to the image reconstructor, the full amount of collected detector data (e.g., all energy bin counts) may be sent to the image reconstructor as well, which may allow for additional images to be reconstructed using all the available (e.g., non-compressed) detector data.

FIG. 5 illustrates a method 500 for identifying target bin combinations to be used to downsample detector bin counts. Method 500 may be carried out according to instructions stored in memory of one or more controllers or computing devices included as part of and/or operatively coupled to a CT imaging system, such as DAS 214, X-ray controller 210, image reconstructor 230, and/or computing device 216. In some examples, method 500 may be carried out as part of method 400, for example as the identification of target bin combinations of 404 of method 400.

At 502, method 500 includes performing a calibration scan using one or more phantoms. As explained previously, the calibration scan may be performed to calibrate the specific CT imaging system, such as to determine the response of each detector element, identify optimal energy bin thresholds, etc. During the calibration scan, one or more phantoms are scanned so that the material composition and thickness of the imaging subject is known. The phantoms may be composed of one or more suitable materials, such as polyvinyl chloride (PVC) or polyethylene (PE). In some examples, the phantoms may include regions of water, iodide, calcium, and/or other materials (e.g., other contrast agents). In some examples, one or more of the phantoms may be step-wedge phantoms. During the calibration scan, the CT imaging system may be controlled so that the X-ray source emits X-rays which are detected by the detector of the CT imaging system after attenuation by each scanned phantom.

At 504, the full bin counts are obtained from the detector. The full bin counts may be similar to the full bin counts described above with respect to FIG. 4, e.g., photon counts partitioned into the full number of energy bins allowed by the configuration of the detector, such as 5 or 8 bins. Bin counts (e.g., photon counts partitioned into energy bins) may be obtained for each pixel of the detector and for each view obtained during the scan of each phantom. The full bin counts may be sent from the DAS 214 to the image reconstructor 230 and/or the computing device 216.

At 506, one or more target bin combinations are identified. Each target bin combination may specify which energy bins should be combined to downsample the detector data, as described above with respect to FIG. 4. The number of target bin combinations to be identified may be based on the configuration of the CT imaging system, the image reconstruction goal of the scans that will be performed, the scan protocol to be applied for the scans that will be performed, and/or other factors. For example, for scan protocols where three basis materials will be decomposed, three target bin combinations may be identified, while only two target bin combinations may be identified for scan protocols where two basis materials will be decomposed. Further, as will be explained in more detail below, the target bin combinations may be identified that maintain an optimization metric at a target level, and the number of target bin combinations may be based on maintaining the optimization at the target level. For example, three target bin combinations may be identified instead of two target bin combinations because there may be a penalty in the optimization metric when identifying only two target bin combinations.

To identify the target bin combinations, all possible pairs of bins may be iteratively evaluated for impact on an optimization metric, such as contrast to noise ratio (CNR) or MD basis noise in calibration projections (CNR or MD noise in the projections is related to the CNR or MD noise in the reconstructed image and thus may serve as a surrogate metric). Once the best bin pair has been identified, the process may be repeated to identify a next best bin pair and/or to place additional bins into the best bin pair, until the desired number of target bin combinations has been identified. To accomplish this, as indicated at 508, identifying the target bin combinations comprises, for a first pair of bins, combining the bins while keeping the remaining bins separate. For example, bins 1 and 2 may be combined while bins 3-8 are kept separate/as is. An optimization metric is then calculated from the combined bin and separate bins, as indicated at 510. The optimization metric may be calculated or determined from x-ray projection measurements, simulated data, or images. For example, the optimization metric may be calculated or determined from x-ray projection measurements obtained for a calibration phantom, such as a step wedge phantom. For example, a calibration projection measurement could be obtained of a combination of the two basis materials, for example PE and PVC. Material decomposition may be performed on this projection measurement using the combined bin and the remaining separate bins to estimate the thickness of the two basis materials. A metric quantifying the noise in the basis material estimates may be used as the optimization metric. The basis estimates resulting from the combined bin and remaining separate bins may be linearly combined to form a VMI projection estimate. Using the same method, a VMI projection may be estimated for measurements through a different combination of basis materials. The CNR between these two VMI estimates may be used as the optimization metric. An example metric quantifying material decomposition noise is the following equation, where

$${σ}_{B 1}^{2}$$

is the noise in the estimated thickness of the first basis material,

$${σ}_{B 2}^{2}$$

is the noise in the estimated thickness of the second basis material,

$${ℓ}_{B 1}^{2}$$

is the known thickness of the first basis material, and

$${ℓ}_{B 2}^{2}$$

is the known thickness of the second basis material:

$${MD}_{noise} = \frac{{σ}_{B 1}^{2}}{{ℓ}_{B 1}} + \frac{{σ}_{B 2}^{2}}{{ℓ}_{B 2}}$$

As another example, the optimization metric may be calculated from one or more images generated from the combined bin and remaining separate bins. For example, a VMI may be generated for a CT acquisition of a phantom as described above with respect to FIG. 4, and the CNR (e.g., the iodine or bone CNR) of the VMI may be determined as the optimization metric. A combination of the basis noise or VMI CNR metrics could also be used as the optimization metric. The target bin combinations could also be optimized using projections simulated by a model of the imaging system and detector response.

This process is repeated for each remaining pair, as indicated at 512. For example, on a next iteration, bins 1 and 3 may be combined while all remaining bins (including bin 2) are maintained separate, and the optimization metric may be calculated using the combined bin (1 and 3) and the remaining separate bins (2 and 4-8). On the next iteration after that, bins 1 and 4 may be combined, then bins 1 and 5, and so forth, until an optimization metric is calculated for all possible pairs. At 514, the pair of bins with the best optimization metric (e.g., highest CNR or lowest MD noise) is selected and the bins in the pair are combined into a “super” bin.

At 516, the process is again repeated until the desired number of bins is reached. Once a first super bin is created, the bins in the super bin are maintained together but treated as a single bin for the purposes of identifying the bin combinations. For example, if the first iteration identifies a super bin including bins 1 and 2, on the next iteration, bins 1 and 2 may be combined with bin 3 while all other bins remain separate, then bins 1 and 2 may be combined with bin 4 while all other bins remain separate, etc. When bins 3 and 4 are combined, for example, bins 1 and 2 may remain together, as the super bin acts as a single bin. Once each energy bin has been assigned to a super bin and the number of super bins is equal to the desired number of bins, the target bin combinations have been identified, and the target bin combinations are saved in memory (e.g., in DAS 214), as indicated at 518, to be applied during a subsequent scan of an imaging target, such as the scan described above with respect to FIG. 4. In other examples, the iterative process of combining bins and calculating the optimization metric may be repeated until the optimization metric reaches a target level. For example, if three target bin combinations maintain CNR above a target level but two target bin combinations cause CNR to drop below the target level, the three target bin combinations may be selected and saved in memory.

FIG. 15 schematically shows a process 1500 for iteratively combining bins to identify the target bin combinations described above. The process 1500 starts with 8 original bins, shown at 1502. The 8 original bins are iteratively combined into pairs as described above and each pair (in combination with the remaining separate bins) is assessed for the optimization metric. As shown at 1504, the pair of bins that resulted in the best optimization metric (e.g., highest CNR) was bins 1 and 7. Bins 1 and 7 form a super pair that acts as a single bin, and the process is repeated. As shown at 1506, the next pair that results in the best optimization metric is bins 2 and 8, which then form a second super bin. The process is repeated to identify the next pair, which as shown at 1508 is bins 1, 6, and 7 (because bins 1 and 7 act as a single bin, the pair is 1+7 and 6). On the next iteration, shown at 1510, the pair of bins 4 and 5 is identified as a super bin. The process is again repeated (twice) until the final two bins are identified, as shown at 1512.

The identification of the target or super bins may be repeated with different phantoms, in order to identify target bin combinations based on subject thickness, subject material, or other parameters. Further, the target bin combinations may be identified for the detector as a whole, for each view. In other examples, different target bin combinations may be identified for each pixel or region of pixels and/or for each view. For example, the calibration may be performed with a step-wedge phantom, and the optimal target bin combinations can be determined during calibration for different patient thicknesses (which correspond to different parts of the step wedge) as well as for different imaging tasks. As will be explained in more detail below, from the scout scan or from the scan prescription, the patient size can be estimated, and the optimal bin combinations may be selected by matching the patient size to the similar calibration data.

Additionally, in some examples, the calibration data may be used to estimate a respective model for each target bin combination, where each model may be applied to perform material decomposition. In some examples, a model may be estimated for each native energy bin (e.g., eight models may be estimated, one for each energy bin when eight bins are collected). However, material decomposition may be lower quality (e.g., unstable) if these native energy bin models are applied. Instead, quality of the material decomposition may be improved if a model is estimated for each target bin combination rather than each individual bin. In some examples, these models may be used to estimate the MD noise optimization metric.

FIG. 6 illustrates a method 600 for identifying target weight sets to be used to downsample detector bin counts. Method 600 may be carried out according to instructions stored in memory of one or more controllers or computing devices included as part of and/or operatively coupled to a CT imaging system, such as DAS 214, X-ray controller 210, image reconstructor 230, and/or computing device 216. In some examples, method 600 may be carried out as part of method 400, for example as the identification of target weight sets of 406 of method 400.

At 602, method 600 includes performing a calibration scan using one or more phantoms. As explained previously, the calibration scan may be performed to calibrate the specific CT imaging system, such as to determine the response of each detector element, identify optimal energy bin thresholds, etc. During the calibration scan, one or more phantoms are scanned so that the material composition of the imaging subject is known. The phantoms may be composed of one or more suitable materials, such as PVC or PE. In some examples, the phantoms may include regions of water, iodide, calcium, and/or other materials (e.g., other contrast agents). In some examples, one or more of the phantoms may be step-wedge phantoms. As a non-limiting example, the step-wedge phantom may be a 2D step wedge phantom that includes 0 to 40 cm of water with step spacing of 4 cm and 0 to 2 cm of calcium with step spacing of 0.2 cm in the orthogonal direction. During the calibration scan, the CT imaging system may be controlled so that the X-ray source emits X-rays which are detected by the detector of the CT imaging system after attenuation by each scanned phantom.

At 604, the full bin counts are obtained from the detector. The full bin counts may be similar to the full bin counts described above with respect to FIG. 4, e.g., photon counts partitioned into the full number of energy bins allowed by the configuration of the detector, such as 5 or 8 bins. Bin counts (e.g., photon counts partitioned into energy bins) may be obtained for each pixel of the detector and for each view obtained during the scan of each phantom. The full bin counts may be sent from the DAS 214 to the image reconstructor 230 and/or the computing device 216.

At 606, a forward model is defined for the detector based on the full bin counts. The forward model may be used for performing maximum likelihood estimation of material decomposition from binned (or weighted binned) counts, as will be explained in more detail below. The forward model may account for (and correct) the energy response and beam hardening effects in each bin.

A semi-empirical polychromatic Beer-Lambert model may be applied to describe the detected counts in each energy bin, which can successfully capture the non-ideal detector energy response and beam hardening effects in each bin. In some examples, the forward model and the corresponding parameters may include:

$${λ}_{i} = {λ}_{0 , i} {e}^{- {\sum }_{m = 1}^{M} {t}_{m} {μ}_{m} ( {\hat{E}}_{i} )} + {\sum }_{k = 1}^{K} {λ}_{k , e} {e}^{- {\sum }_{m = 1}^{M} {c}_{k , i , m} {t}_{m} {μ}_{m} ( {\hat{E}}_{i} )} ,$$

where λᵢ is the photon counts detected in energy bin i, tₘ is the known line integral of the mth basis material, μₘ(Êᵢ) is the linear attenuation coefficient of the corresponding basis material, and Êᵢ is the effective energy of bin i for the model. This model uses additional correction terms to correct the energy response and beam hardening effects in each bin. For each energy bin, with M basis materials and K correction terms, there are K(M+1)+2 system parameters to estimate. Then for mean observed counts yᵢ (tₙ) in energy bin i

$${λ}_{0 , i} , {λ}_{k , i} , {\hat{E}}_{i} , {c}_{k , i , m} = {arg min}_{{λ}_{0 , i} , {λ}_{k , i} , {\hat{E}}_{i} , {c}_{k , i , m}} {\sum }_{n = 1}^{N} {λ}_{i} ( {t}_{n} ) - {y}_{i} ( {t}_{n} ) log {λ}_{i} ( {t}_{n} ) ,$$

where tₙ=(t_{Calcium,n}, t_{Water,n}) is the known line integral of the two basis materials in the nth calibration data point.

To further improve this semi-empirical forward model, the ratio yᵢ(t)/λᵢ(t) may be fit with a 2nd-degree polynomial function ƒ(t), so that the expected binned photon counts can be expressed as \tilde{λ}ᵢ(t)=λᵢ(t)ƒ(t). This semi-empirical forward model can then be used for maximum likelihood estimation of material decomposition from binned (or weighted binned) counts.

At 608, method 600 includes identifying two or more target weight sets. Each target weight set may specify an amount by which each energy bin should be weighted prior to summing the weighted bin counts to downsample the detector data, as described above with respect to FIG. 4. The number of target weight sets to be identified may be based on the configuration of the CT imaging system, the image reconstruction goal of the scans that will be performed, the scan protocol to be applied for the scans that will be performed, and/or other factors. For example, for scan protocols where three basis materials will be decomposed, three target weight sets may be identified, while only two target weight sets may be identified for scan protocols where two basis materials will be decomposed.

Identifying the target weight sets may include searching through continuous weight space to find two target weight sets, as indicated at 610. In general, the weights can take on any continuous real value. The weights may be normalized to a range of ±1 since normalization, scaling, or linearly independent combinations of the weights produce weights with which the summed weighted bin counts contain the same amount of information. In some examples, the weights may be binary weights, which are a special case of the more general continuous weights. With binary weights, the weights are either 0 or 1, and each original bin either does not or does contribute to the weighted bins through a summation. The contribution from the original bins may be mutually exclusive, in which case each original bin contributes once and only once to a summed bin. The target weight sets may be identified from among all possible weight sets based on which two weight sets minimize the ratio between the variance of basis material thickness estimated from the sets of projection data for different object materials and thicknesses. Accordingly, each pair of weight sets (from all possible weight sets) may be used to weight the full/original bin counts, and the respective weighted bin counts may be summed. As indicated at 612, for each pair of weight sets applied to the bin counts, the forward model identified at 610 is applied to estimate the basis material thickness, and as indicated at 614, the pair of weight sets that minimize the material thickness estimate variance are identified as the target weight sets (in this case, the ratio between the Cramer-Rao lower bound (CRLB) of basis material thickness estimates from the weighted sums and that from the original binned counts). A similar approach may be applied if the desired number of bins to be formed is three bins instead of two, in which case the basis material thickness is determined for each set of three weight sets (from all possible weight sets) and the set of three weight sets that minimizes the material thickness estimate variance is selected as the target weight sets.

As a specific example, two sets of weights, W_{i,j}, may be identified, such that weighted sums b_{Wj}=ΣᵢW_{i,j}bᵢ contain similar spectral information to the original binned counts bᵢ, where j indexes the two sets of weights and i indexes the original energy bins. To make the noise performance of material decomposition estimates from the two weighted measurements comparable to that from the original binned counts, the ratio between the variance of basis material thickness estimated from these two sets of projection data for different object materials and thicknesses is minimized. Because the maximum likelihood estimator is asymptotically unbiased, the analytically calculated Cramér-Rao lower bound (CRLB) may be used instead to form the objective function:

$$\hat{W} = {arg min}_{W} {\sum }_{t} \frac{{CRLB}_{W} ( Ca , Ca )}{CRLB ( Ca , Ca )} {\vert}_{t} + \frac{{CRLB}_{W} ( water , water )}{CRLB ( water , water )} {\vert}_{t} + \frac{{CRLB}_{W} ( {VMI}_{60 keV} )}{CRLB ( {VMI}_{60 keV} )} {\vert}_{t} ,$$

where CRLB_{W}(Ca, Ca) and CRLB_{W}(water, water) are the lower bound of the variance of the estimated calcium and water thicknesses, respectively, from an unbiased estimator with two weighted bin measurements generated with weights W and CRLB_{W}(VMI_{60keV}) is the variance for the corresponding virtual monoenergetic image at 60 keV; and CRLB(Ca, Ca), CRLB(water, water), and CRLB(VMI_{60keV}), are the corresponding lower bound of the variance estimated from the original binned counts. To obtain the analytical CRLB expression, an independent Poisson distribution may be assumed for the original binned counts, and a multivariate Gaussian distribution may be assumed for energy-weighted measurements.

Once the target weight sets have been identified, the target weight sets are saved in memory (e.g., in DAS 214), as indicated at 618, to be applied during a subsequent scan of an imaging target, such as the scan described above with respect to FIG. 4.

The identification of the target weight sets may be performed with the step-wedge phantom as described above, with the target weight sets identified that minimize the CRLB for all combinations of material thicknesses of the step-wedge phantom. However, additionally or alternatively, the process described above may be repeated with different phantoms or different target weight sets may be identified for different combinations of material thickness of the step-wedge phantom, in order to identify target weight sets based on subject thickness, subject material, or other parameters. Further, the target weight sets may be identified for the detector as a whole, for each view. In other examples, different target weight sets may be identified for each pixel or region of pixels and/or for each view.

FIG. 7 illustrates a method 700 for downsampling detector bin counts. Method 700 may be carried out according to instructions stored in memory of one or more controllers or computing devices included as part of and/or operatively coupled to a CT imaging system, such as DAS 214, X-ray controller 210, image reconstructor 230, and/or computing device 216. In some examples, method 700 may be carried out as part of method 400, for example as the downsampling of bin counts at 410 of method 400.

At 702, method 700 includes determining scan parameters to be applied during the scan of the imaging subject. The scan parameters may be determined based on a selected scan protocol and/or based on user input received at a computing device of the CT imaging system (e.g., computing device 216). The scan parameters may include the scan prescription (e.g., X-ray source voltage and current, slice thickness, gantry table speed, etc.), the anatomy being scanned, whether one or more contrast agents have been administered to the imaging subject, and other parameters.

At 704, method 700 determines if the scan parameters dictate that uniform downsampling should be performed. Uniform downsampling may include applying the same target weight sets or target bin combinations to all collected detector data. Non-uniform downsampling may include applying different target weight sets or target bin combinations to different aspects of the detector data, such as to different views.

If uniform downsampling is indicated, method 700 proceeds to 706 to select the target bin combinations or target weight sets that are optimized for all scans. As explained above with respect to FIGS. 5 and 6, during calibration, target bin combinations or target weight sets may be identified. These target bin combinations or target weight sets may be identified to optimize image reconstruction for all patient sizes. Alternatively or additionally, different target bin combinations or different target weight sets may be identified based on material thickness, based on detector element/pixel, and/or based on view. When uniform downsampling is applied, the target bin combinations or target weight sets identified that optimize image reconstruction for all scans may be selected. As a non-limiting example, the computing device 216 may instruct the DAS 214 to downsample the detector data using the selected target bin combinations or target weight sets or the computing device 216 may indicate to the DAS 214 that uniform downsampling is to be applied and the DAS 214 may select the target bin combinations or target weight sets.

At 708, method 700 includes, for each pixel or detector element of the detector, combine the full/original bin counts using the target combinations or the target weight sets, to form the downsampled bin counts. In this way, the detector data from each pixel may be compressed from the full number of bins (e.g., eight or five bins) to a reduced number of bins (e.g., two or three bins). When the bins are compressed using the target bin combinations, the bins in each combination may be summed. For example, if the target bin combinations specify that bins 1, 2, 3, and 7 be combined into a first super bin, bins 4 and 5 be combined into a second super bin, and bin 8 be left as is, bins 1, 2, 3, and 7 are summed and bins 4 and 5 are summed (for each pixel). When the bins are compressed using the target weight sets, each bin may be weighted by an amount specified by a weight of a first weight set and then the weighted bins may be summed to form a first weighted summed bin (for each pixel). Each original bin may be weighted by an amount specified by a weight of a second weight set and then the weighted bins may be summed to form a second weighted summed bin (for each pixel).

At 710, the downsampled bin counts are sent (e.g., from the DAS) to the image reconstructor (or another suitable computing device), where the downsampled bin counts are usable to reconstruct one or more images, as explained above with respect to FIG. 4. In some examples, as indicated at 712, the original/full bin counts are also sent to the image reconstructor. In this way, initial images may be generated relatively quickly using the downsampled bin counts, while additional images may be generated using the original/full bin counts after a delay period (as the original/full bin counts may take more time to be sent to the image reconstructor), if desired.

Returning to 704, if it is determined that uniform downsampling is not to be performed (e.g., the downsampling is to be non-uniform), method 700 proceeds to 714 to determine a size of the imaging subject from a scout scan image, user input, and/or detector counts. A scout scan may be a low dose scan that results in one or more low resolution images usable to confirm correct positioning of the imaging subject, set the scan prescription, or other functions. The image(s) reconstructed from the projection data collected during the scout scan may be analyzed to determine the size (e.g., thickness) of the imaging subject. Additionally or alternatively, the size of the imaging subject may be determined from user input and/or based on the detector counts themselves (e.g., lower detector counts may indicate a thicker imaging subject).

At 716, the target bin combinations or target weight sets are selected based on the size of the imaging subject. As explained above, during calibration, different target bin combinations or different target weight sets may be identified based on material thickness, based on detector element/pixel, and/or based on view. When non-uniform downsampling is applied, the target bin combinations or target weight sets identified that optimize image reconstruction for material thickness equal to or in range of the imaging subject size/thickness may be selected. As a non-limiting example, the computing device 216 may instruct the DAS 214 to downsample the detector data using the selected target bin combinations or target weight sets or the computing device 216 may send the subject size information to the DAS 214 and the DAS 214 may select the target bin combinations or target weight sets based on the subject size information. In some examples, as indicated at 718, selecting the target bin combinations or target weight sets based on subject size may include selecting the targets in a view by view manner, which may also be based on subject thickness. For example, bin counts obtained from the detector for a first view may be downsampled with first target bin combinations or first target weight sets while bin counts obtained from the detector for a second, different view may be downsampled with second target bin combinations or second target weight sets (different than the first). This view by view target selection may be applied because subject thickness at the edges of the subject may be less than subject thickness at the center of the subject. Further, as indicated at 720, selecting the target bin combinations or target weight sets based on subject size may include selecting the targets in a detector element by detector element manner (e.g., by pixel or regions or pixels), which may or may not be based on subject thickness. For example, bin counts obtained from a first element of the detector may be downsampled with first target bin combinations or first target weight sets while bin counts obtained from a second, different detector element of the detector may be downsampled with second target bin combinations or second target weight sets (different than the first). This element by element target selection may account for differences in the response of different detector elements. Method 700 then proceeds to 708 to combine the bin counts using the target bin combinations or target weight sets, as explained above.

Thus, method 700 provides for the selection and application of target bin combinations or target weight sets, which may be applied uniformly to all bin counts or may be applied differentially based on subject size, CT imaging system configuration, user preference, or other factors. For example, a scan protocol may dictate that a child is being imaged and thus first bin combinations or weight sets may be applied, while a different scan protocol may dictate that an adult is being imaged and thus second, different bin combinations or weight sets may be applied. It is to be appreciated that the view by view target selection or the element by element target selection may be performed without taking into regard subject thickness, in some examples.

FIG. 8 shows a set of graphs 800 depicting example energy spectra of combined bins, where the bins are combined using the target bin combinations described herein (e.g., with respect to FIG. 5). A first graph 810 shows the energy spectra for three bins combined according to target bin combinations identified using grayscale image CNR as the optimization metric and imaging a phantom with material composition of 20 cm water and 1 cm of iodine (at 5 mg/mL) as well as PVC and PE. The first graph 810 depicts the number of photons detected as a function of the photon energy for three combined bins. The combined bins include combined bin 1, where the original bins 4, 5, and 6 are combined; combined bin 2, where the original bins 1, 2, 3, and 7 are combined, and a combined bin 3, including only bin 8. The downsampled bins, when used to reconstruct images, resulted in a slight decrease in the CNR (e.g., −4%) but a relatively large increase the MD basis noise for both PVC and PE (an increase of 59% and 52%, respectively).

A second graph 820 shows the energy spectra for three bins combined according to target bin combinations identified using MD basis noise as the optimization metric and imaging a phantom with material composition of 20 cm water and 1 cm of iodine (at 5 mg/mL) as well as PVC and PE. The second graph 820 depicts the number of photons detected as a function of the photon energy for three combined bins. The combined bins include combined bin 1, where the original bins 4 and 5 are combined; combined bin 2, where the original bins 1, 2, 3, 7, and 8 are combined, and a combined bin 3, including only bin 6. Thus, when optimizing for MD basis noise rather than grayscale CNR, the target bin combinations may be different than when optimizing for CNR. The downsampled bins, when used to reconstruct images, resulted in a slight decrease in the CNR (e.g., −7%, which is more than when CNR is the optimization metric) and also decreases in the MD basis noise for both PVC and PE (a decrease of 0.5% and 13%, respectively).

FIG. 9 shows a graph 900 of the energy spectra for three bins combined according to target bin combinations identified using grayscale image CNR as the optimization metric and imaging a phantom with material composition of 20 cm water and 1 cm of iodine (at 5 mg/mL) as well as PVC and PE. In contrast to the first graph of FIG. 8, the graph 900 shows energy spectra for bins combined with an additional requirement that the bins that are combined be contiguous bins. The graph 900 depicts the number of photons detected as a function of the photon energy for three combined bins. The combined bins include combined bin 1, where the original bins 4, 5, 6, and 7 are combined; combined bin 2, where the original bins 1, 2, and 3 are combined, and a combined bin 3, including only bin 8. The downsampled bins, when used to reconstruct images, resulted in a larger decrease in the CNR (e.g., −8%) than that exhibited when non-contiguous bins are allowed to be combined. As such, obtaining the full 8 bins and compressing the bins using the target bin combinations described herein (where non-contiguous bins can be combined) results in better quality images than simply reducing the number of bins that are obtained.

FIG. 8 shows energy spectra and corresponding CNR and MD basis noise for bins combined according to the above-described target bin combinations where the bin counts were obtained while imaging a phantom having a first amount of water (e.g., 20 cm). Similar analysis was conducted on bins combined according to the target bin combinations while imaging a phantom having a second amount of water (e.g., 40 cm). For the target bin combinations optimized for grayscale CNR, when imaging the phantom having the second amount of water, the CNR reduction was the same as described above while PE exhibited a noise increase of 53% and PVC a noise increase of 58%. For the target bin combinations optimized for MD basis noise, when imaging the phantom having the second amount of water, the CNR reduction was 11% while PE exhibited a noise increase of 2% and PVC a noise increase of 15%. Thus, at least for bin combinations optimized for MD noise, variation in image quality based on material thickness may be present, which may be mitigated by selecting different bin combinations for different material thicknesses.

FIG. 10 shows a set of virtual monoenergetic images 1000 reconstructed from eight acquired energy bins (as shown by first image 1002) and three energy bins combined using the target bin combinations described herein (as shown by second image 1004). Both images demonstrated nearly equivalent CNR between the bone and soft tissue regions marked in the images and equivalent CNR between the soft tissue and adipose regions. Both images represent 68 keV VMI and are displayed at a window of −1000 to 1800 HU.

FIG. 11 shows a graph 1100 illustrating a target weight set that may be applied to downsample bin counts (e.g., according to the method of FIG. 6) and FIG. 12 shows a graph 1200 of an example corresponding energy spectra of the bins created by applying the target weight set. Graph 1100 shows a set of optimal weights W (unfilled boxes and filled boxes) for a silicon detector plotted against the corresponding effective energy of each bin. Note that these weights are normalized to 1 and any linearly independent combination of them will yield the same results. In the example shown, the set of weights includes a first weight set shown by the unfilled boxes (to be applied to bins 1-8, respectively) and a second weight set shown by the filled boxes (to be applied to bins 1-8, respectively). To form a first weighted summed bin, the weights in the first weight set may be applied to each respective bin (e.g., a first weight W_{(1,1)} applied to the bin count of bin 1, a second weight W_{(1,2)} applied to the bin count of bin 2, and so forth until an eighth weight W_{(1,8)} is applied to the bin count of bin 8) and the weighted bin counts are summed. To form a second weighted summed bin, the weights in the second weight set may be applied to each respective bin (e.g., a ninth weight W_{(2,1)} applied to the bin count of bin 1, a tenth weight W_{(2,2)} applied to the bin count of bin 2, and so forth) and the weighted bin counts are summed.

Graph 1200 shows the energy spectra for an example scan of an imaging subject as the number of photons detected as a function of the photon energy for the two weighted summed bins formed from the target weight sets of the graph 1100.

FIG. 13 shows a first set of MD basis images 1300 of a phantom reconstructed from full bin data (e.g., 8 bins) and compressed bin data (e.g., 2 bins), where the bin counts are compressed using the target weight sets as described herein. A first image 1302 shows a calcium MD basis image using the compressed bin counts (e.g., the two energy weighted bins) and a second image 1304 shows a calcium MD basis image using the full 8 bins. A common ROI is magnified for both images. A first magnified ROI 1306 shows the ROI for the first image 1302 while a second magnified ROI 1308 shows the ROI for the second image 1304. An ROI difference image 1310 visually depicts the differences between the two ROIs. As appreciated from the images shown in FIG. 13, the image reconstructed using the two energy-weighted bins is nearly identical to the image reconstructed using the full 8 bins, with a mean variance penalty of the ROI of 11.57%.

FIG. 14 shows a second set of MD basis images 1400 of a phantom reconstructed from full bin data (e.g., 8 bins) and compressed bin data (e.g., 2 bins), where the bin counts are compressed using the target weight sets as described herein. A first image 1402 shows a water MD basis image using the compressed bin counts (e.g., the two energy weighted bins) and a second image 1404 shows a water MD basis image using the full 8 bins. A common ROI is magnified for both images. A first magnified ROI 1406 shows the ROI for the first image 1402 while a second magnified ROI 1408 shows the ROI for the second image 1404. An ROI difference image 1410 visually depicts the differences between the two ROIs. As appreciated from the images shown in FIG. 14, the image reconstructed using the two energy-weighted bins is nearly identical to the image reconstructed using the full 8 bins, with a mean variance penalty of the ROI of 5.55%.

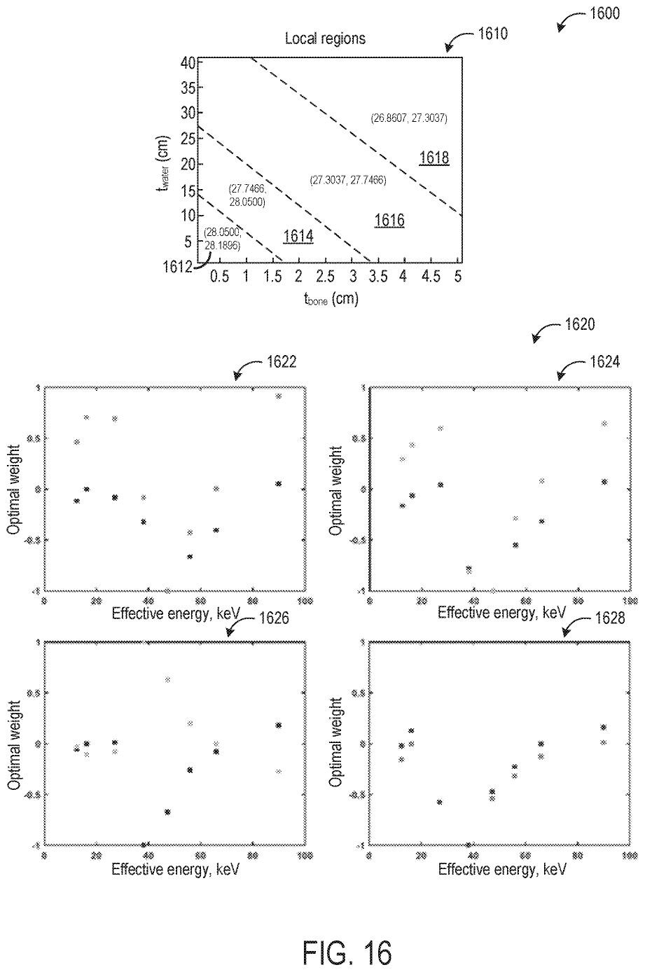

FIG. 16 shows example weight sets that may be applied to downsample bin counts in a localized manner, such as based on a size of an imaging subject. FIG. 16 includes a first graph 1610 showing four regions defined by bone thickness (increasing along the x-axis in graph 1610) and water thickness (increasing along the y-axis in graph 1610). For different bone and water thickness combinations, the corresponding attenuated X-ray spectra will be different, which results in different distributions of the original bin counts data as well as different effective energy of the spectra, which is defined by

$${E}_{eff} ( {t}_{Bone} , {t}_{Water} ) = \frac{{\sum }_{i = 1}^{{N}_{bins}} \frac{{U}_{low}^{( i )} + {U}_{high}^{( i )}}{2} {N}_{i} ( {t}_{Bone} , {t}_{Water} )}{{\sum }_{i = 1}^{{N}_{bins}} {N}_{i} ( {t}_{Bone} , {t}_{Water} )} ,$$

where t_{Bone} is the thickness of bone and t_{Water} is the thickness of water,

$${U}_{low}^{( i )} and {U}_{high}^{( i )}$$

are the corresponding lower and higher threshold of the ith original energy bin, N_{bins} is the number of original energy bins, and Nᵢ(t_{Bone}, t_{Water}) is the detected photon counts in the ith original energy bin when the x-ray spectrum is attenuated by an object composed of bone and water with thickness t_{Bone}, t_{Water}. The ranges of bone and water thicknesses of graph 1610 are divided into a first region 1612, a second region 1614, a third region 1616, and a fourth region 1618 by dividing this whole material space into uniformly spaced values of E_{eff}. The numbers in each region indicate the lower and higher bound of values of E_{eff} that define the local regions.

FIG. 16 also includes a set of graphs 1620 showing example weights that may be applied to downsample bin counts, with each graph from the set of graphs 1620 corresponding to a different region of graph 1610. For example, graph 1622 shows first weight sets that may be applied to downsample bin counts obtained while scanning a subject having the material thicknesses defined by region 1618 (e.g., a relatively large/thick subject), graph 1624 shows second weight sets that may be applied to downsample bin counts obtained while scanning a subject having the material thicknesses defined by region 1616 (e.g., a medium to large/thick subject), graph 1626 shows third weight sets that may be applied to downsample bin counts obtained while scanning a subject having the material thicknesses defined by region 1614 (e.g., a small to medium sized subject), and graph 1628 shows fourth weight sets that may be applied to downsample bin counts obtained while scanning a subject having the material thicknesses defined by region 1612 (e.g., a relatively small subject). As explained above, subject size/thickness may be determined based on one or more images acquired during the scout scan, based on user input and/or a scan protocol, and/or based on the detector counts themselves.

Thus, energy bins may be combined according to the methods disclosed herein in order to compress/downsample the amount of data sent from the CT machine to an off-board computing device/image reconstructor. The energy bins may be combined using a weighted approach where each energy bin is weighted and summed with the remaining weighted energy bins. Two or more weight sets may be applied to form two or more combined bins. The weighting may be binary such that each bin is either included or not included in a combined bin. This binary approach may be exclusionary, such that each bin is included once and only once in a combined bin. Such an approach results in the different target bin combinations described herein (e.g., with respect to FIG. 5). In other examples, the binary approach may not be exclusionary, and bins may be included in more than one combined bin. In still further examples, the weighting approach may include the application of continuous weights, as explained herein with respect to FIG. 6, for example. Identifying the continuous weights may include identification and application of a forward model and large-scale optimization, which may be computationally intensive relative to identifying binary weights. However, at least in some examples, the continuous weights may result in higher image quality. As such, the selection of which weighting approach to apply in order to downsample the detector data/energy bins may be based on specific imaging tasks that will be applied, computational power available, and/or desired image quality.

The technical effect of downsampling energy bin photon counts from a full amount of energy bins to a reduced number of energy bins is that less data is transmitted to an external computing device for reconstruction (e.g., across the slip ring of the CT system), thereby expediting the process of reconstructing images from the energy bin photon counts. Another technical effect of acquiring a full number of energy bin counts (e.g., 8 energy bins) and then downsampling the full number of bins into a reduced number of bins is that the target number of combined/downsampled bins (e.g., 2 or 3 combined bins) may change based on patient size, the task of interest, region of the image that is of interest for the task, and other factors. By acquiring the full number of bins natively, the bins may be combined in different ways for different purposes after the fact. Further, if the target bin combination includes non-contiguous bins (which may result in better image quality than combining only contiguous bins due to characteristics of the energy bins that detect Compton scatter), the target bin combinations may not be possible to acquire natively. Additionally, the full set of native energy bins may still be transferred later and used to benefit reconstructions that are less time sensitive.

In another representation, a method for a photon counting computed tomography (PCCT) system is provided, the method comprising: during a calibration phase, obtaining detector data from a detector of the PCCT system while scanning an object of known material composition and thickness, the detector data comprising, for each pixel of the detector, photon counts partitioned into a plurality of energy bins based on an energy of each photon that impinges on the detector, identifying, based on the detector data, one or more bin factors to be applied in a subsequent scan to downsample subsequent detector data into a reduced number of energy bins, during the subsequent scan, obtaining the subsequent detector data and downsampling the subsequent detector data into the reduced number of energy bins by applying at least one of the one or more bin factors, and reconstructing one or more images from the downsampled subsequent detector data. In a first example of the method, identifying, based on the detector data, one or more bin factors comprises identifying one or more target bin combinations that specify which energy bins of the plurality of energy bins are to be combined to form the reduced number of energy bins. In a second example of the method, optionally including the first example, identifying the one or more target bin combinations comprises: iteratively combining pairs of energy bins, determining an optimization metric for each combined pair of energy bins based on images reconstructed from each combined pair of energy bins and remaining separate energy bins, selecting the pair of energy bins with the best optimization metric and setting the selected pair as a single super bin, and repeating the iteratively combining, determining, and selecting until the reduced number of energy bins is reached. In a third example of the method, optionally including one or both of the first and second examples, the optimization metric comprises contrast to noise ratio of virtual monoenergetic images or material decomposition noise of material decomposition basis images. In a fourth example of the method, optionally including one or more or each of the first through third examples, identifying, based on the detector data, one or more bin factors comprises identifying one or more target weight sets that each specify a respective weight to be applied to each energy bin of the plurality of energy bins to form weighted bins which are summed to form the reduced number of energy bins. In a fifth example of the method, optionally including one or more or each of the first through fourth examples, identifying the one or more target weight sets comprises: determining a forward model for the detector based on the detector data, for each pair of weight sets of a plurality of possible weight sets, applying that pair of weight sets to the plurality of energy bins to form two weighted summed bins, applying the forward model to the two weighted summed bins to estimate basis material thickness, and selecting the pair of weight sets that minimize a variance in the estimated basis material thickness.

The disclosure also provides support for a method for a photon counting computed tomography (PCCT) system, the method comprising: during a scan of an imaging subject, obtaining detector data from a photon counting detector of the PCCT system, the detector data comprising, for each pixel or detector element of the photon counting detector, photon counts partitioned into a plurality of energy bins based on an energy imparted by each photon on the photon counting detector, applying a bin factor to the plurality of energy bins for each pixel to downsample the plurality of energy bins into a reduced number of energy bins, and reconstructing one or more images from the reduced number of energy bins. In a first example of the method, applying the bin factor to the plurality of energy bins comprises combining the plurality of energy bins into the reduced number of energy bins according to target bin combinations that specify which energy bins of the plurality of energy bins are to be combined. In a second example of the method, optionally including the first example, combining the plurality of energy bins into the reduced number of energy bins according to target bin combinations includes combining at least two non-contiguous bins. In a third example of the method, optionally including one or both of the first and second examples, applying the bin factor to the plurality of energy bins comprises applying two or more target weight sets to the plurality of energy bins to form two or more sets of weighted bins and summing each set of weighted bins to form the reduced number of energy bins. In a fourth example of the method, optionally including one or more or each of the first through third examples, the method further comprises: identifying the bin factor based on calibration detector data obtained from a calibration scan of a phantom, the calibration detector data comprising, for each pixel or detector element of the photon counting detector, photon counts partitioned into a plurality of energy bins based on an energy of each photon that impinges on the detector. In a fifth example of the method, optionally including one or more or each of the first through fourth examples, identifying the bin factor based on the calibration detector data comprises identifying one or more target bin combinations that specify which energy bins of the plurality of energy bins are to be combined to form the reduced number of energy bins, and wherein identifying the one or more target bin combinations comprises: iteratively combining pairs of energy bins, determining an optimization metric for each combined pair of energy bins based on x-ray projection measurements, simulated data, or images reconstructed from each combined pair of energy bins and remaining separate energy bins, selecting the pair of energy bins with the best optimization metric and setting the selected pair as a single super bin, and repeating the iteratively combining, determining, and selecting until the reduced number of energy bins is reached. In a sixth example of the method, optionally including one or more or each of the first through fifth examples, the optimization metric comprises contrast to noise ratio of virtual monoenergetic images or material decomposition noise of material decomposition basis images. In a seventh example of the method, optionally including one or more or each of the first through sixth examples, identifying the bin factor based on the calibration detector data comprises identifying one or more target weight sets that each specify a respective weight to be applied to each energy bin of the plurality of energy bins to form weighted bins which are summed to form the reduced number of energy bins, and wherein identifying the one or more target weight sets comprises: determining a forward model for the detector based on the calibration detector data, for each pair of weight sets of a plurality of possible weight sets, applying that pair of weight sets to the plurality of energy bins to form two weighted summed bins, applying the forward model to the two weighted summed bins to estimate basis material thickness, and selecting the pair of weight sets that minimize a variance in the estimated basis material thickness or virtual monoenergetic images. In an eighth example of the method, optionally including one or more or each of the first through seventh examples, applying the bin factor to the plurality of energy bins for each pixel to downsample the plurality of energy bins into the reduced number of energy bins comprises applying the bin factor to downsample five or eight energy bins into two or three energy bins. In a ninth example of the method, optionally including one or more or each of the first through eighth examples, the same bin factor is applied to downsample the plurality of energy bins for each pixel and each view of the detector data. In a tenth example of the method, optionally including one or more or each of the first through ninth examples, different bin factors are applied to downsample the plurality of energy bins for different pixels and/or different views of the detector data. In an eleventh example of the method, optionally including one or more or each of the first through tenth examples, the bin factor is selected based on a size of the imaging subject. In an eleventh example of the method, optionally including one or more or each of the first through tenth examples, the pixel is a detector element of the photon counting detector.

The disclosure also provides support for a photon counting computed tomography (PCCT) system, comprising: an X-ray source that emits a beam of X-rays toward a subject to be imaged, a photon counting detector that receives the beam of X-rays attenuated by the subject, and a data acquisition system (DAS) operably connected to the photon counting detector and configured to: during a scan of an imaging subject, obtain detector data from the photon counting detector, the detector data comprising, for each pixel or detector element of the photon counting detector, photon counts partitioned into a plurality of energy bins based on an energy imparted by each photon that impinges on the photon counting detector, apply a bin factor to the plurality of energy bins for each pixel to downsample the plurality of energy bins into a reduced number of energy bins, and send, via a slip ring of the PCCT system, the reduced number of energy bins to a computer comprising non-transitory memory and operably connected to the DAS, wherein the computer is configured with instructions in the non-transitory memory that when executed cause the computer to reconstruct one or more images from the reduced number of energy bins. In a first example of the system, applying the bin factor to the plurality of energy bins comprises combining the plurality of energy bins into the reduced number of energy bins according to target bin combinations that specify which energy bins of the plurality of energy bins are to be combined. In a second example of the system, optionally including the first example, applying the bin factor to the plurality of energy bins comprises applying two or more target weight sets to the plurality of energy bins to form two or more sets of weighted bins and summing each set of weighted bins to form the reduced number of energy bins. In a third example of the system, optionally including one or both of the first and second examples, the pixel is a detector element of the photon counting detector.

This disclosure also provides support for a method for a photon counting X-ray imaging system, the method comprising: during a scan of an imaging subject, obtaining detector data from a photon counting detector of the X-ray imaging system, the detector data comprising, for each pixel of the photon counting detector, photon counts partitioned into a plurality of energy bins based on an energy imparted by each photon on the photon counting detector; applying a bin factor to the plurality of energy bins for each pixel to downsample the plurality of energy bins into a reduced number of energy bins; and reconstructing one or more images from the reduced number of energy bins.

This disclosure also provides support for a photon counting X-ray imaging system, comprising: an X-ray source that emits a beam of X-rays toward a subject to be imaged; a photon counting detector that receives the beam of X-rays attenuated by the subject; and a data acquisition system operably coupled to the photon counting detector and configured to: during a scan of an imaging subject, obtain detector data from the photon counting detector, the detector data comprising, for each pixel of the photon counting detector, photon counts partitioned into a plurality of energy bins based on an energy imparted by each photon on the photon counting detector; apply a bin factor to the plurality of energy bins for each pixel to downsample the plurality of energy bins into a reduced number of energy bins; and send the reduced number of energy bins to a computer comprising non-transitory memory and operably coupled to the data acquisition system, wherein the computer is configured with instructions in the non-transitory memory that when executed cause the computer to reconstruct one or more images from the reduced number of energy bins.

Though a photon counting computed tomography (PCCT) system is described by way of example, it should be understood that the present techniques may also be useful when applied to other X-ray imaging modalities having photon counting detectors, such as X-ray angiography systems, X-ray tomosynthesis systems, X-ray mammography systems, X-ray fluoroscopy systems, X-ray interventional systems, X-ray C-arm systems, etc. The present discussion of a PCCT imaging modality is provided merely as an example of one suitable imaging modality.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method for a photon counting computed tomography (PCCT) system, the method comprising:
during a scan of an imaging subject, obtaining detector data from a photon counting detector of the PCCT system, the detector data comprising, for each pixel of the photon counting detector, photon counts partitioned into a plurality of energy bins based on an energy imparted by each photon on the photon counting detector;
applying a bin factor to the plurality of energy bins for each pixel to downsample the plurality of energy bins into a reduced number of energy bins; and
reconstructing one or more images from the reduced number of energy bins.

2. The method of claim 1, wherein applying the bin factor to the plurality of energy bins comprises combining the plurality of energy bins into the reduced number of energy bins according to target bin combinations that specify which energy bins of the plurality of energy bins are to be combined.

3. The method of claim 2, wherein combining the plurality of energy bins into the reduced number of energy bins according to target bin combinations includes combining at least two non-contiguous bins.

4. The method of claim 1, wherein applying the bin factor to the plurality of energy bins comprises applying two or more target weight sets to the plurality of energy bins to form two or more sets of weighted bins and summing each set of weighted bins to form the reduced number of energy bins.

5. The method of claim 1, further comprising identifying the bin factor based on calibration detector data obtained from a calibration scan of a phantom, the calibration detector data comprising, for each pixel of the photon counting detector, photon counts partitioned into a plurality of energy bins based on an energy of each photon that impinges on the detector.

6. The method of claim 5, wherein identifying the bin factor based on the calibration detector data comprises identifying one or more target bin combinations that specify which energy bins of the plurality of energy bins are to be combined to form the reduced number of energy bins, and wherein identifying the one or more target bin combinations comprises:
iteratively combining pairs of energy bins;
determining an optimization metric for each combined pair of energy bins based on x-ray projection measurements, simulated data, or images reconstructed from each combined pair of energy bins and remaining separate energy bins;
selecting the pair of energy bins with the best optimization metric and setting the selected pair as a single super bin; and
repeating the iteratively combining, determining, and selecting until the reduced number of energy bins is reached.

7. The method of claim 6, wherein the optimization metric comprises contrast to noise ratio of virtual monoenergetic images or material decomposition noise of material decomposition basis images.

8. The method of claim 5, wherein identifying the bin factor based on the calibration detector data comprises identifying one or more target weight sets that each specify a respective weight to be applied to each energy bin of the plurality of energy bins to form weighted bins which are summed to form the reduced number of energy bins, and wherein identifying the one or more target weight sets comprises:
determining a forward model for the detector based on the calibration detector data;
for each pair of weight sets of a plurality of possible weight sets, applying that pair of weight sets to the plurality of energy bins to form two weighted summed bins;
applying the forward model to the two weighted summed bins to estimate basis material thickness; and
selecting the pair of weight sets that minimize a variance in the estimated basis material thickness or virtual monoenergetic images.

9. The method of claim 1, wherein applying the bin factor to the plurality of energy bins for each pixel to downsample the plurality of energy bins into the reduced number of energy bins comprises applying the bin factor to downsample five or eight energy bins into two or three energy bins.

10. The method of claim 1, wherein the same bin factor is applied to downsample the plurality of energy bins for each pixel and each view of the detector data.

11. The method of claim 1, wherein different bin factors are applied to downsample the plurality of energy bins for different pixels and/or different views of the detector data.

12. The method of claim 1, wherein the bin factor is selected based on one or more of the following factors, individually or in combination, comprising:
a size or thickness of the imaging subject;
a projection view;
a detector element or a detector region;
a detector response characteristic;
a material composition of the imaging subject;
an imaging task;
a scan protocol; and
a CT imaging system configuration.

13. The method of claim 1, wherein the pixel is a detector element of the photon counting detector.

14. A photon counting computed tomography (PCCT) system, comprising:
an X-ray source that emits a beam of X-rays toward a subject to be imaged;
a photon counting detector that receives the beam of X-rays attenuated by the subject; and
a data acquisition system (DAS) operably connected to the photon counting detector and configured to:
during a scan of an imaging subject, obtain detector data from the photon counting detector, the detector data comprising, for each pixel of the photon counting detector, photon counts partitioned into a plurality of energy bins based on an energy imparted by each photon on the photon counting detector;
apply a bin factor to the plurality of energy bins for each pixel to downsample the plurality of energy bins into a reduced number of energy bins; and
send, via a slip ring of the PCCT system, the reduced number of energy bins to a computer comprising non-transitory memory and operably connected to the DAS, wherein the computer is configured with instructions in the non-transitory memory that when executed cause the computer to reconstruct one or more images from the reduced number of energy bins.

15. The PCCT system of claim 14, wherein applying the bin factor to the plurality of energy bins comprises combining the plurality of energy bins into the reduced number of energy bins according to target bin combinations that specify which energy bins of the plurality of energy bins are to be combined.

16. The PCCT system of claim 14, wherein applying the bin factor to the plurality of energy bins comprises applying two or more target weight sets to the plurality of energy bins to form two or more sets of weighted bins and summing each set of weighted bins to form the reduced number of energy bins.

17. The PCCT system of claim 14, wherein the pixel is a detector element of the photon counting detector.

18. A method for a photon counting X-ray imaging system, the method comprising:
during a scan of an imaging subject, obtaining detector data from a photon counting detector of the X-ray imaging system, the detector data comprising, for each pixel of the photon counting detector, photon counts partitioned into a plurality of energy bins based on an energy imparted by each photon on the photon counting detector;
applying a bin factor to the plurality of energy bins for each pixel to downsample the plurality of energy bins into a reduced number of energy bins; and
reconstructing one or more images from the reduced number of energy bins.

19. A photon counting X-ray imaging system, comprising:
an X-ray source that emits a beam of X-rays toward a subject to be imaged;
a photon counting detector that receives the beam of X-rays attenuated by the subject; and
a data acquisition system operably coupled to the photon counting detector and configured to:
during a scan of an imaging subject, obtain detector data from the photon counting detector, the detector data comprising, for each pixel of the photon counting detector, photon counts partitioned into a plurality of energy bins based on an energy imparted by each photon on the photon counting detector;
apply a bin factor to the plurality of energy bins for each pixel to downsample the plurality of energy bins into a reduced number of energy bins; and
send the reduced number of energy bins to a computer comprising non-transitory memory and operably coupled to the data acquisition system, wherein the computer is configured with instructions in the non-transitory memory that when executed cause the computer to reconstruct one or more images from the reduced number of energy bins.
